(19) United States
(12) Patent Application Publication
(10) Pub. No.: US 2007/0146635 A1
(43) Pub. Date: Jun. 28, 2007

(54) PUPIL REFLECTION EYE TRACKING SYSTEM AND ASSOCIATED METHODS

(76) Inventors: Richard A. LeBlanc, Clermont, FL (US); Martin Sensiper, Orlando, FL (US); Thomas L. McGilvary JR., Oviedo, FL (US)

Correspondence Address: ALCON, IP LEGAL, TB4-8, 6201 SOUTH FREEWAY, FORT WORTH, TX, 76134, US

(21) Appl. No.: 11/615,384

(22) Filed: Dec. 22, 2006

Related U.S. Application Data
(60) Provisional application No. 60/753,157, filed on Dec. 22, 2005.

(51) Int. Cl. A61B 3/10

(52) U.S. Cl. 351/221

(57) ABSTRACT

A system for tracking eye movement includes a detector that is adapted to receive radiation reflected from a retina defining a spatial extent of a pupil of an eye. The detector acts to generate data indicative of a positioning of the received radiation on the detector. A processor is in communication with the detector and has software resident thereon for determining from an analysis of the data a pupil position. A controller is in communication with the processor and with a device for adjusting a direction of radiation emitted by an illumination source responsive to the determined pupil position in order to substantially center the emitted radiation on the pupil. The illumination source is preferably coaxial with the detector, and emits a beam having a diameter less than the pupil diameter.

### CROSS-REFERENCE TO RELATED APPLICATION

This application claims priority under 35 U.S.C. § 119 to U.S. Provisional Patent Application No. 60/753,157 filed Dec. 22, 2005, the entire contents of which are incorporated herein by reference.

### FIELD OF THE INVENTION

The invention relates generally to optical tracking systems, and more particularly to optical systems for tracking pupil position.

### BACKGROUND OF THE INVENTION

In an ophthalmic surgical procedure, unwanted eye movement can degrade the outcome of the surgery. Eye positioning is critical in such procedures as corneal ablation, since a treatment laser is typically centered on the patient's theoretical visual axis which, practically speaking, is approximately the center of the patient's pupil. However, this visual axis is difficult to determine due in part to residual and involuntary eye movement. Therefore, it is critical to stabilize the eye with respect to the surgical apparatus for best outcomes.

Previous disclosure of eye tracking systems and methods has been made, for example, in U.S. Pat. Nos. 5,980,513; 6,315,773; and 6,451,008, which are co-owned with the present application, and which are hereby incorporated by reference hereinto. Video and LADAR tracking are also known in the art. Most known systems for tracking an eye require a specular reflection from the cornea as a reference, which cannot be used in LASIK-type surgeries, since the smooth surface of the cornea is replaced with a rougher surface when the stroma is exposed by flap cutting. Video trackers have been shown to work for this purpose, but these are not robust against unusual eyes. Further, these systems tend to be relatively expensive, as they require high-speed cameras and high-speed processing capabilities. Further, the trackers known to be used at the present time are not known to be successful with small, undilated pupils and intraocular lenses.

Therefore, it would be desirable to provide a system and method for tracking eyes, for example, during a surgical procedure, without relying on corneal properties, and also capable of functioning on pupils in an undilated condition.

### SUMMARY OF THE INVENTION

The present invention is useful for tracking eye movement by using the eye's retroreflecting properties and a detector, and can be used on dilated and undilated eyes. For small-spot refractive surgery systems, stabilizing the eye is critical for best outcomes. This is typically performed with the use of an eye tracker. A successful tracker has two phases of operation: acquisition and tracking. While tracking is characterized by keeping a particular object in a specific spot relative to a known reference, acquisition is characterized by finding the object within a search volume. If acquisition is not successful, either the tracker will not engage, or will track the wrong object.

A system for tracking eye movement comprises a detector that is adapted to receive radiation reflected from a retina through a pupil of an eye. The detector acts to generate data indicative of a positioning of the received radiation on the detector. A processor is in communication with the detector and has software resident thereon for determining from an analysis of the data a pupil position. A controller is in communication with the processor and with means for adjusting a direction of radiation emitted by an illumination source responsive to the determined pupil position in order to substantially center the emitted radiation on the pupil. Preferably the illumination source substantially coaxial with the detector and is configured to emit a beam of radiation having a diameter less than a pupil diameter.

A method of the present invention includes the step of receiving on a detector radiation reflected from retina through a pupil of an eye. Data indicative of a positioning of the received radiation on the detector are generated, and a pupil position is determined from an analysis of the data. A direction of radiation emitted by an illumination source is then able to be adjusted responsive to the determined pupil position in order to substantially center the emitted radiation on the pupil.

This technique may be used on objects other than corneas, and in surgical procedures other than corneal ablation.

An important feature of the present invention is that it is not intended for use with a so-called “bright pupil.” Rather, what is intended to be detected is a pupil “glow,” which is unfocused radiation projected onto the retina and detected on the cornea. There are substantially no data impinging on the detector relating to external eye structure or features other than pupil size. Ideally, the radiation reflected should form a step function, with all radiation received at the detector from the pupil and the area surrounding the pupil contributing no data. In reality, of course, it is difficult to achieve a completely “on/off” data set, since the pupil boundary will not be on exact pixel boundaries, so that some pixels will have an intermediate value due to being only partially illuminated. To address this, a threshold is set below which the data are considered to have a zero value.

### BRIEF DESCRIPTION OF THE DRAWINGS

FIG. 1 is an exemplary geometry for a quadrant detector for use with the present invention.

FIG. 2 is a schematic diagram of an eye tracking system using polarized light.

FIG. 3 is a schematic diagram of an eye tracking system using unpolarized light.

FIG. 4 is a schematic diagram of an eye tracking system using an imaging focal plane detector.

FIG. 5 is a schematic diagram of an eye tracking system using polarized beams.

FIG. 6 is a schematic diagram of a particular embodiment of the system of FIG. 5 with the laser in the pass direction of the beam splitter.

FIG. 7 is a schematic diagram of a particular embodiment of the system of FIG. 5 with the detector in the pass direction of the beam splitter.

FIG. 8 is a schematic diagram of an eye tracking system using a collimation lens and beam shaping optics.

FIG. 9 is a schematic diagram of a particular embodiment of the system of FIG. 8 using a beam expander.

FIG. 10 is a schematic diagram of a particular embodiment of the system of FIG. 8 using high-numerical-aperture focusing optics.

FIGS. 11A-11E and 12A-12E are two series of images taken as a laser spot is scanned across the pupil, with FIGS. 11A-11E taken with a CMOS camera and FIGS. 12A-12E taken with a camera sensitive only to the laser wavelength.

FIG. 13 is an exemplary intensity scan taken across a pupil in two dimensions, showing the zero crossing at the pupil centroid.

### DETAILED DESCRIPTION OF THE PREFERRED EMBODIMENTS

The present invention will now be described with reference to FIGS. 1-13.

A system and method for tracking transverse movement comprise a pupil tracking device that uses “pupil glow” to determine the center of the pupil for the purpose of maintaining an ablating laser beam in a preferred orientation relative to the cornea.

A particular embodiment of the system 10 includes a quadrant detector 11 (FIG. 1) that is adapted to receive radiation reflected 12 from a retina 13 through a pupil 14 of an eye 15 (FIGS. 2 and 3), the reflected radiation 12 initiated by emitted radiation 16 sent to the pupil 14 from an illumination source 17. Although the illumination source 17 can in principle emit in any wavelength range that can enter and be reflected from the retina of the eye 15, it is believed preferable that the illumination source 17 emit in the infrared, more preferably, in the near-infrared, and, most preferably, below 1.5 μm. The illumination source 17 can be pulsed, modulated, or continuous wave, depending upon the noise that is expected from other parts of the system 10. The illumination source 17 can also comprise a monochromatic laser, a light-emitting diode (LED), a superluminescent LED, a resonant-cavity LED, or a conventional light source that is filtered and focused.

An important feature of the system 10 is that the illumination source is adapted to emit a beam of radiation that has a diameter less than a pupil diameter, for example, 1 mm, although this is not intended to be limiting. Thus the beam 16 can be directed to impinge on and be completely surrounded by the pupil 14 when centered properly, so that substantially all emitted radiation 16 is sent into the eye 15. Further, such a beam 16 will result in detectable reflected radiation 12 in all types of eyes, even those that are significantly disparate from emmetropic.

The detector 11 can comprise, for example, a quadrant detector that is divided into quarters and has a plurality of concentric, substantially toroidal zones 18-20 subdivided into quarter-sectors 18a-18d, etc., having a center 21. In a particular embodiment, the detector 11 comprises a high-sensitivity quadrant detector sensitive to all wavelengths usable for illumination of an eye. The zones 18-20 are used depending upon the size of the pupil 14, with the inner zones 18 used for smaller pupil sizes, etc., as will be described in the following.

The detector 11 is used to generate data indicative of a positioning of the received radiation on the detector 11, these data then sent to a processor 23 having software 24 resident thereon for determining from an analysis of the data a pupil position.

A controller 25 is in communication with the processor 23 and with means for adjusting a direction of radiation emitted by the illumination source 17 responsive to the determined pupil position in order to substantially center the emitted radiation on the pupil 13.

Preferably the system 10 further comprises a beamsplitter that is positioned to reflect radiation from the illumination source 17 onto the eye 15 and to pass the reflected radiation 12 to the detector 11, for permitting a substantially coincident path of the emitted radiation 16 and the reflected radiation 12.

In a first embodiment 10 (FIG. 2), the illumination source 17 is polarized 26, and the beamsplitter comprises a polarizing beamsplitter 27. This configuration permits the beamsplitter 27 to select from the pupil glow and the specular reflections from the surface of the cornea 28.

In a second embodiment 10′ (FIG. 3), the illumination source is unpolarized, and the beamsplitter 27′ is also unpolarized. In this configuration, it is preferable to mask 29 specular reflection from the eye 15 from reaching the detector 11. Such a mask 29 will be positioned at the center 30 of the detector 11, since such specular reflection will normally be centered.

In order that refractive errors be minimized, a zoom element 31 can be positioned upstream of the detector 11 for maintaining an image of the pupil 13 at the detector 11 at a substantially constant size. Such a zoom element 31 can comprise, for example, a true zoom, a step zoom, or a true zoom with detents. In some systems a zoom may not be required.

The processor 23 is used to process detector data, select the zone(s) to use, and create an error signal based upon the ratios of the signals in the zones. The processor 23 then controls via the controller 25 optical elements 32 such as mirrors positioned downstream of the illumination source 17 and upstream of the pupil 14. The optical elements 32 are used to stabilize the image on the detector 11 so that the emitted beam 16 is maintained close to the center of the eye 15, so that the image can be stabilized on a display.

Although not intended to be limiting, the quadrant detector 11 can be used as follows: In FIG. 1, the hatched area 33 represents a circle of reflected radiation from an eye 15. An efficient data analysis method comprises, for each quarter, determining an outermost quarter-sector containing reflected radiation and analyzing the data in that outermost quarter-sector only. In the example shown in FIG. 1, quarter-sectors 18a-18d are completely covered by the hatched area 33, and are not considered in the analysis. Assuming that the pupil 14 is circular, the data in quarter-sectors 19a,19b,20c,20d would be sufficient to determine the hatched area's center 34, with additional data from quarter-sectors 19c, 19d completing the circle if necessary and/or desired.

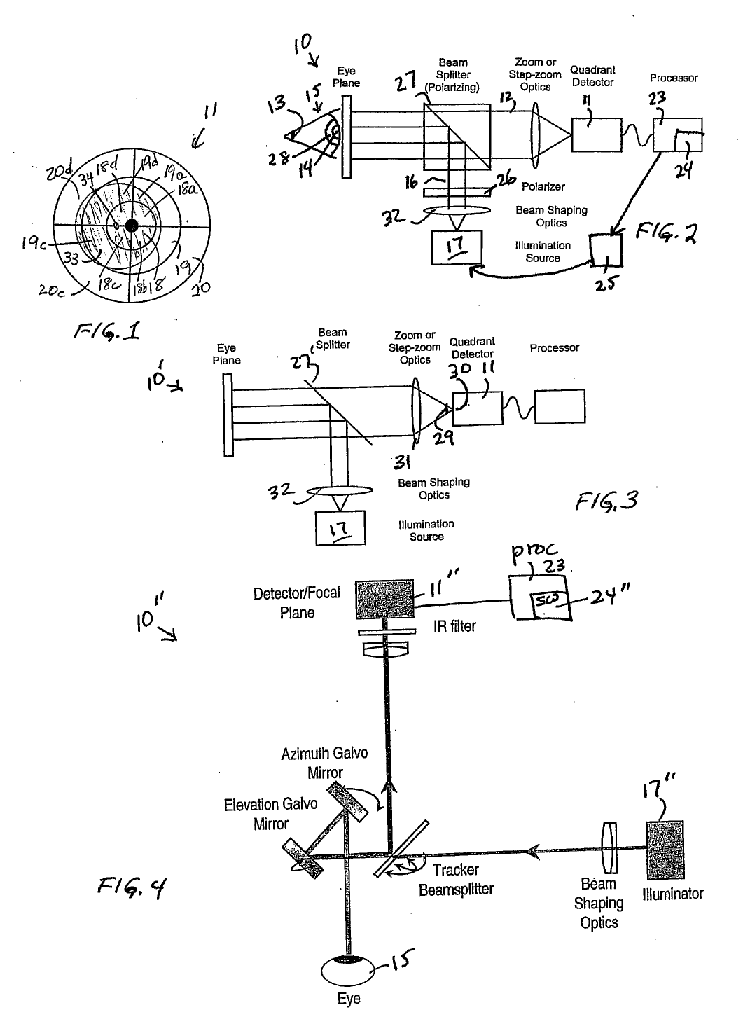

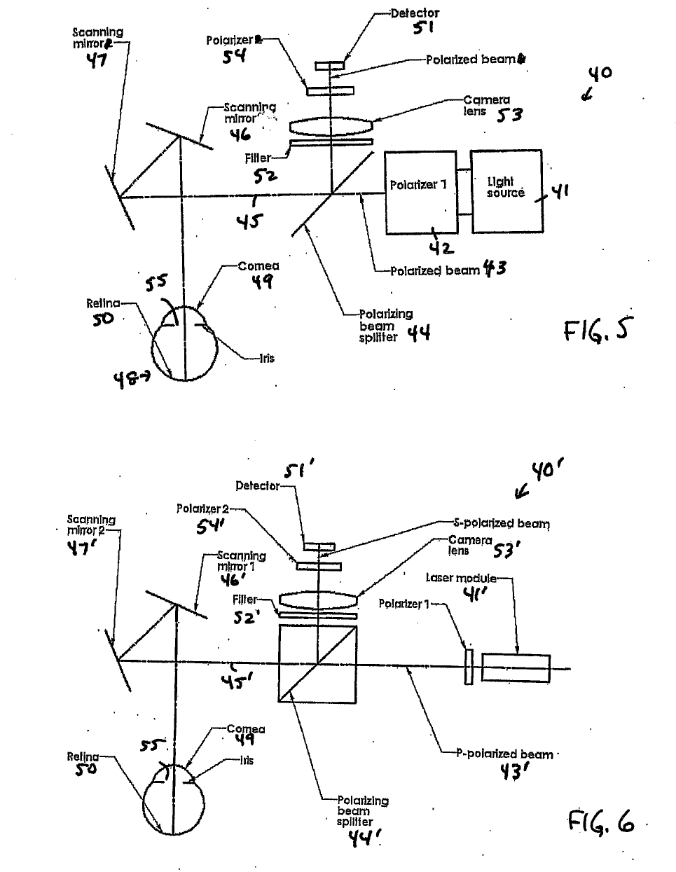

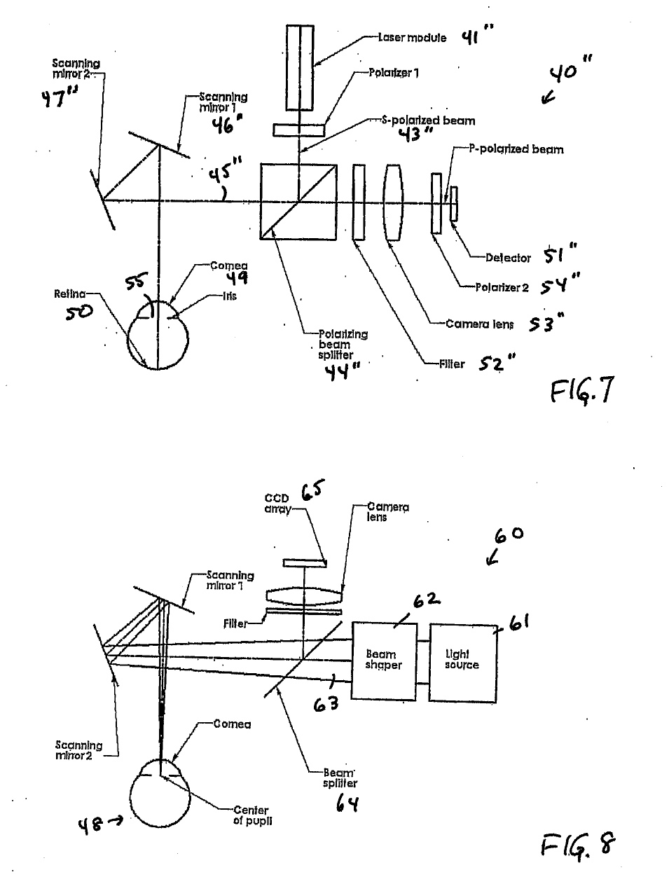

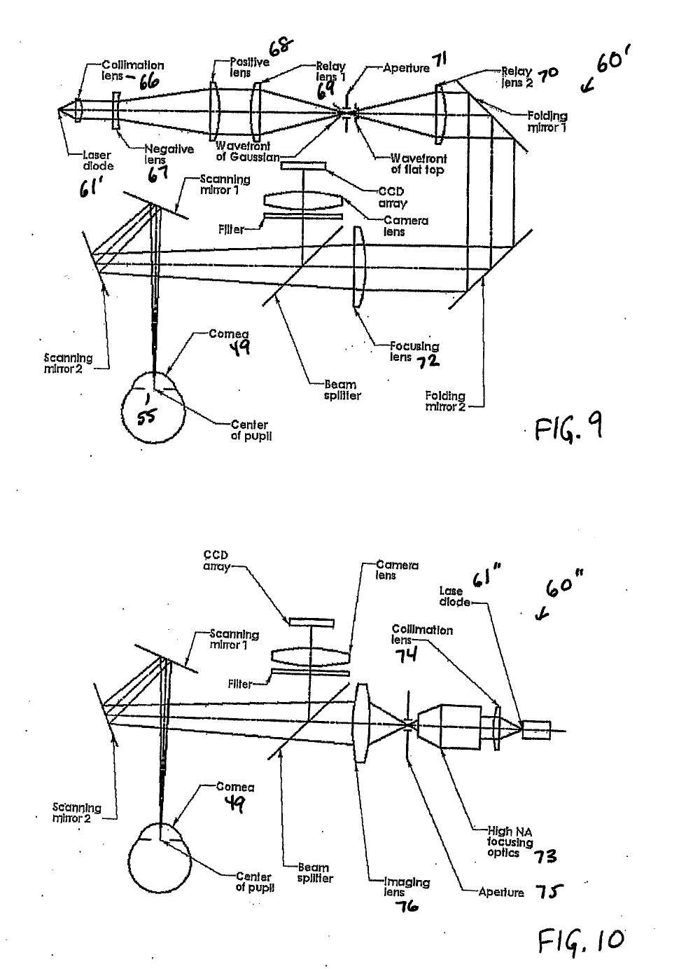

In another embodiment 10″ (FIG. 4), the detector 11″ comprises a high-speed imaging detector that is positioned at a focal plane of the illumination source 17″, which can be unpolarized. In this embodiment 10″, the generated data comprise pixel data, with the software 24″ adapted to determine from the pixel data the pupil's position geometrically. The detector 11″ can comprise, for example, a complementary metal oxide semiconductor (CMOS) sensor having a windowing capability, although this is not intended as a limitation. Here a non-contiguous windowing capability can be used to realize a zoned concept.

In an imaging system 10″, the data can be reduced to a minimum complexity, and the detector 11″ can be used in a non-imaging mode. The focal plane imager can calculate substantially the same error signal as with the quadrant detector 11 from the discrete pixels in a digital (on/off) fashion. The CMOS detector can reduce processing to a minimum. In one method, for example, the pixels can be counted as in/not in the pupil, and the pupil geometry can be derived as an area centroid.

Here the system 10″ thresholds the image, and the specular reflection issue is obviated, since such reflections are interior to the pupil and the intensity of the reflection is “masked” by the binary nature of the thresholding decision.

If a zoom is used, a variable-dimension subframe window can be used as the zoomed image.

In a particular embodiment, the beamsplitter can comprise a mirror having a central hole therein. The mirror can be placed so that the hole has negligible effect on the image, but passes substantially all the illumination energy. This provides close to 100% laser transmission, which allows a smaller laser to be used. On the receive side, there are no “ghost” images from the two sides of the beamsplitter, permitting virtually 100% transmission, thereby reducing the illumination requirements. Such a mirror can have a diameter of approximately 25-30 mm, for example, and the hole, 3 mm diameter.

In video-based pupil tracking systems that use unpolarized light, the illumination light reflected from the cornea has a much higher intensity compared with the pupil area illuminated by light scattered from the retina. Since the cornea-reflected light may be an order of magnitude stronger than the pupil area light, any direct transmitting, internal reflections, and stray light may significantly alter the irradiance map of the pupil image in the detector. Therefore, it would desirable to eliminate unwanted light from corneal reflection.

A general schematic diagram (FIG. 5) of another configuration 40 for the present invention includes a light source 41 sent through a polarizer 42 to produce a polarized beam 43 that in turn proceeds to a polarizing beam splitter (PBS) 44. This configuration 40 eliminates reflected light the from cornea. The part 45 of the beam 43 that is transmitted through the beam splitter 44 is routed via two scanning mirrors 46,47 to the eye 48. When polarized light is incident on an eye 48, a portion of light reflects back from the cornea 49, while the other portion of the light enters the eye 48 and is scattered from the retina 50. The light reflected from the cornea 49 keeps the polarization direction of the incident light, while the light scattered from the retina 50 becomes unpolarized. The return beam is reflected by scanning mirrors 46,47. The polarizing beam splitter 44 blocks the polarized light from the corneal reflection so that only light from the retina 50 can reach the detector 51, which in this embodiment is preceded by a filter 52, camera lens 53, and second polarizer 54. Approximately one-half of the unpolarized light emitted by the pupil area 55 reaches the detector 51.

In an embodiment 40′ (FIG. 6) of the configuration 40 of FIG. 5, the beam 43′ comprises a p-polarized beam. The laser module 41′ can comprise, for example, a laser diode and a collimation/focusing lens. The PBS 44′ passes the p-polarized light and reflects s-polarized light. The p-polarized light 45′ exiting from the PBS 44′ is reflected by the scanning mirrors 46′,47′. A portion of the light incident on the cornea 49 is reflected by the cornea 49 and remains p-polarized. This cornea-reflected light is further reflected by the scanning mirrors 46′,47′ and passes through the PBS 44′. Another portion of the light incident on the cornea 49 goes through the cornea 49 and is scattered by the retina 50. The pupil 55 is illuminated by retina-scattered light that is unpolarized. Light from the pupil area 55 is reflected by scanning mirrors 46′,47′ and is incident on the PBS 44′. s-polarized light is reflected by the PBS 44′ and passes through the filter 52′, camera lens 53′, and second polarizer 54′, and forms an image of the pupil 55 on the detector 51′. This image has a high signal-to-noise ratio, since corneal reflected light has been substantially eliminated.

In another embodiment 40″ (FIG. 7) of the configuration 40 of FIG. 5, the beam 43″ comprises an s-polarized beam. The PBS 44″ passes the s-polarized light and reflects p-polarized light. The s-polarized light 45″ exiting from the PBS 44″ is reflected by the scanning mirrors 46″,47″. A portion of the light incident on the cornea 49 is reflected by the cornea 49 and remains s-polarized. This cornea-reflected light is further reflected by the scanning mirrors 46″,47″ and passes through the PBS 44″. Another portion of the light incident on the cornea 49 goes through the cornea 49 and is scattered by the retina 50. The pupil 55 is illuminated by retina-scattered light that is unpolarized. Light from the pupil area 55 is reflected by scanning mirrors 46″,47″ and is incident on the PBS 44″. p-polarized light is reflected by the PBS 44″ and passes through the filter 52″, camera lens 53″, and second polarizer 54″, and forms an image of the pupil 55 on the detector 51″. Here the laser module 41″ is configured in a reflection direction of the PBS 44″ while the detector is in the pass direction of the PBS 44″.

In other embodiments, the illumination and imaging beams can be cross-circularly polarized.

Typically beams emerging from an illumination source are Gaussian shaped. When such a beam reaches the cornea/pupil area, for a small pupil, especially with a flap, some portion of the beam is also reflected by the iris owing to the tail of the Gaussian beam, thus reducing contrast between the pupil and the iris. For small pupils, this may cause serious tracking errors. Therefore, it would be desirable for the illumination beam to be confined inside the pupil area.

A general schematic diagram (FIG. 8) of another configuration 60 for the present invention includes a light source 61 sent through a beam shaper 62 to produce a beam 63 having a steeper edge than that which emerges from the light source 61. The beam shaper 62 can comprise diffractive or refractive optical components, or spatial light modulators (SLMs). The shaped beam 63 in turn proceeds to a beam splitter (BS) 64 and then in similar fashion to the eye 48, from which pupil glow light returns through the beam splitter 64 and to the detector 65, here shown as a CCD array, although this is not intended as a limitation. The optics between the beam splitter 64 and the eye 48 are substantially the same as those discussed above.

In an embodiment 60′ (FIG. 9) of the configuration 60 of FIG. 8, the laser module 61′ can comprise, for example, a laser diode with a collimating lens 66 in front thereof. The collimated beam is expanded by a beam expander formed by a negative lens 67 and a positive lens 68. The expanded beam then passes through a relay system comprising a first 69 and a second 70 relay lens. A small aperture 71 is placed near the focal position of the first relay lens 69. Following this aperture 71, the incoming Gaussian-shaped beam is transformed into a flat-topped beam, which is then collimated by the second relay lens 70 and focused by a focusing lens 72 onto the cornea/pupil position 49. Thus the pupil 55 is illuminated by a flat-topped beam with a steep edge rather than a Gaussian beam, thereby substantially eliminating return from the iris.

In another embodiment 60″ (FIG. 10) of the configuration 60 of FIG. 8, high-numerical-aperture (NA) focusing optics 73 is employed to replace the beam expander 67,68 in FIG. 9. The high-NA focusing optics 73 can comprise microscope objectives, aspherical lenses, GRIN lenses, and diffractive elements, although these are not intended as limitations. The light emitted by the laser diode 61″ is collimated by a collimating lens 74. The collimated beam then passes through the high-NA focusing optics 73. A small aperture 75 is placed at the focal plane of the focusing optics 73. Following the aperture 75, the edge of the wavefront becomes steep. An imaging lens 76 then forms the image of the aperture 75 onto the pupil position 49.

Another aspect of the present invention is directed to the acquisition of the pupil for tracking using pupil glow. The system of the invention can acquire the pupil in less than 0.5 sec. In this aspect, the illumination beam is scanned over the eye at a very rapid rate, completing the scan in less than 0.5 sec. The illumination beam of the pupil glow tracker is much smaller than the pupil in most cases; the pupil is typically larger than 2 mm, while the illumination beam is approximately 0.5 mm. However, reflections of the beam from various parts of the eye, such as a tear layer or flap bed, can expand the apparent size of the beam on the detector; so size alone is not an adequate discriminator for acquiring a pupil. The shape of the beam can assist in the process, since a reflection from a tear layer will typically not be symmetrical around the beam. However, the diffuse scatter from the flap bed will typically create a circular pattern that can be mistaken for a glowing pupil.

There is one phenomenon that only appears by illuminating a pupil. When the illumination beam just crosses the edge of the pupil, the entire pupil glows. This creates a large error between the pointing position of the beam and the centroid of the return energy. Using this phenomenon, there is a strong probability that the pupil is being illuminated, and that its center is near the centroid calculated. Further processing can be performed to verify that the shape is nearly circular and that the size is stable and of a magnitude that is acceptable for a pupil. This system does not rely on the pupil's stability, and is effective with pupils that are less than four times the beam diameter.

Since a flap creates a noncircularity in the pupil shape as sensed, and since an opaque bubble layer in the interior of the cornea can scatter light that hinders detection of the pupil glow, the boundary of the pupil can be determined as far as possible, and then a circular shape can be extrapolated from the determined boundary. If the determined boundary is insufficiently circular, the system can indicate that the entity being acquired is not in fact the pupil, and tracking must be repeated.

In FIGS. 11A-11E are displayed a sequence of images taken with a CMOS camera as a laser spot is scanned across a pupil. The calculated centroid is shown beneath each image. The camera and the reference spot are fixed in the same reference field, so that, when the laser spot moves, the camera field of view moves with it. In this way, the return from the laser spot is normally composed of direct energy except when it illuminates the pupil, in which case it is composed of indirect energy (pupil glow).

If the images of FIGS. 11A-11E are viewed by a camera sensitive only to the laser wavelength, the image sequence would look as in FIGS. 12A-12E. As the laser spot is scanned over the pupil from bottom right to top left, the pupil is clearly seen to be illuminated. When the spot first enters the pupil, the calculated centroid is at a maximum and decreases as the spot moves over the pupil until it reaches the center. It then steadily increases until the other edge of the pupil is reaches, where the calculated centroid is again at a maximum.

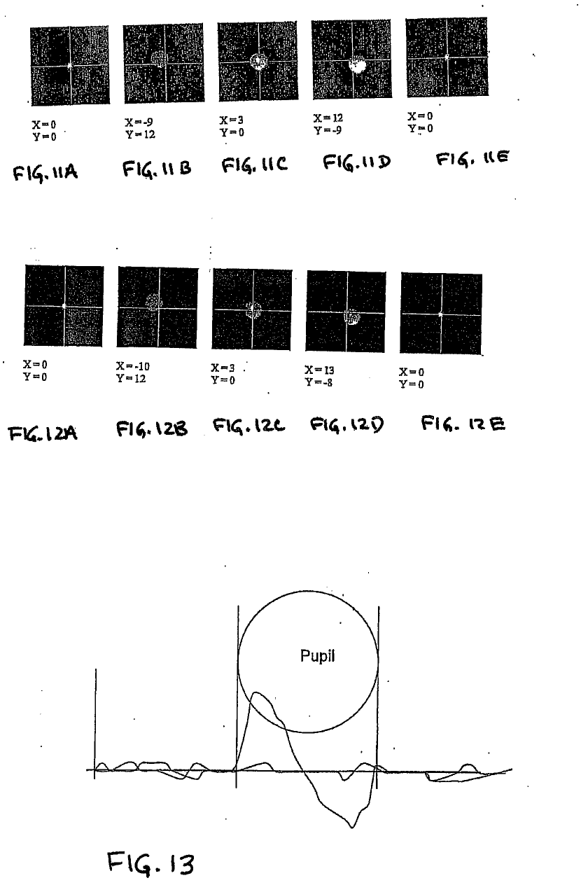

Further, the images in FIGS. 11A, 12A, 11E, and 12E show that the calculated centroid from the spot illuminating the cornea outside the pupil is seen to be very near zero. It is this difference that is used to sense the presence of a pupil. This phenomenon can be used in tracker acquisition by scanning the eye at a high speed and comparing each calculated centroid to a predetermined threshold value known to reliably predict the presence of a pupil. Once this threshold is tripped (see FIG. 13), then the tracker will stop scanning and close a track loop around the current image centroid.

Processing of the image data can optimize the image intensity and the “in/out of pupil” threshold. The threshold can be set based upon the intensity of the pupil by adjusting the camera gain and then adjusting the threshold on the pupil during acquisition, and typically will comprise the half-way point between dark and maximum intensity. During the tracking phase, the beam and the threshold are tracked to keep the intensity of the pupil substantially the same. This system can be adaptive to conditions and to the particular patient.

Jitter detection can also be added to assess tracking for small pupils. Such jitter is typically caused by the hardware, and not by the eye, and can be assessed by tracking the stability of an image.

Although the invention has been described relative to specific embodiments thereof, there are numerous variations and modifications that will be readily apparent to those skilled in the art in the light of the above teachings. It is therefore to be understood that, within the scope of the appended claims, the invention may be practiced other than as specifically described.

What is claimed is:

1. A system for tracking eye movement comprising:
a detector adapted to receive reflected radiation from a retina defining a spatial extent of a pupil of an eye and to generate data indicative of a positioning of the received radiation on the detector;
a processor in communication with the detector having software resident thereon for determining from an analysis of the data a pupil position; and
a controller in communication with the processor and with means for adjusting a direction of radiation emitted by an illumination source responsive to the determined pupil position in order to substantially center the emitted radiation on the pupil, the illumination source substantially coaxial with the detector and configured to emit a beam of radiation having a diameter less than a pupil diameter.

2. The system recited in claim 1, wherein the illumination source is adapted to emit in the infrared range.

3. The system recited in claim 2, wherein the illumination source is adapted to emit below 1.5 μm.

4. The system recited in claim 1, wherein the illumination source is selected from a group consisting of a monochromatic laser, a light-emitting diode, and superluminescent light-emitting diode, and a resonant-cavity light-emitting diode.

5. The system recited in claim 1, further comprising a beamsplitter positioned to reflect radiation from the illumination source onto the eye and to pass the reflected radiation to the detector, for permitting a substantially coaxial path of the emitted radiation and the reflected radiation.

6. The system recited in claim 5, wherein the illumination source is polarized, and wherein the beamsplitter comprises a polarizing beamsplitter.

7. The system recited in claim 1, wherein the illumination source is unpolarized, and further comprising means for masking specular reflection from the eye from reaching the detector.

8. The system recited in claim 1, wherein the illumination source is unpolarized, and the detector comprises an imaging detector positioned at a focal plane of the illumination source, the generated data comprise pixel data, and the software is adapted to determine from the pixel data the pupil position.

9. The system recited in claim 1, further comprising a zoom element positioned upstream of the detector for maintaining an image of the pupil at the detector at a substantially constant size.

10. The system recited in claim 1, wherein the detector comprises a non-imaging detector.

11. The system recited in claim 10, wherein the detector comprises a quadrant detector divided into quarters and having a plurality of concentric, substantially toroidal zones subdivided into quarter-sectors by the quarter divisions.

12. The system recited in claim 1, wherein the detector comprises an imaging detector positioned at a focal plane of the laser, the generated data comprise pixel data, and the software is adapted to determine from the pixel data the pupil position.

13. The system recited in claim 12, wherein the detector comprises a complementary metal oxide semiconductor sensor having a windowing capability.

14. The system recited in claim 1, wherein the adjusting means comprises optics positioned downstream of the illumination source and upstream of the pupil, the optics under control of the controller.

15. A system for tracking eye movement comprising:
a non-imaging detector adapted to receive reflected radiation from a retina defining a spatial extent of a pupil of an eye and to generate data indicative of a positioning of the received radiation on the detector;
a processor in communication with the detector having software resident thereon for determining from an analysis of the data a pupil position;
a controller in communication with the processor and with means for adjusting a direction of radiation emitted by an illumination source responsive to the determined pupil position in order to substantially center the emitted radiation on the pupil, the illumination source configured to emit a beam of radiation having a diameter less than a pupil diameter; and
a beamsplitter positioned to reflect radiation from the illumination source onto the eye and to pass the reflected radiation to the detector, configured for permitting a substantially coaxial path of the emitted radiation and the reflected radiation.

16. The system recited in claim 15, wherein the illumination source is polarized, and wherein the beamsplitter comprises a polarizing beamsplitter.

17. The system recited in claim 15, wherein the illumination source is unpolarized, and further comprising means for masking specular reflection from the eye from reaching the detector.

18. The system recited in claim 15, wherein the illumination source is unpolarized, and the detector comprises an imaging detector positioned at a focal plane of the illumination source, the generated data comprise pixel data, and the software is adapted to determine from the pixel data the pupil position.

19. The system recited in claim 15, further comprising a zoom element positioned upstream of the detector for maintaining an image of the pupil at the detector at a substantially constant size.

20. The system recited in claim 15, wherein the detector comprises a quadrant detector divided into quarters and having a plurality of concentric, substantially toroidal zones subdivided into quarter-sectors by the quarter divisions.

21. A method for tracking eye movement comprising the steps of:
receiving on a detector radiation reflected from retina defining a spatial extent of a pupil of an eye;
generating data indicative of a positioning of the received radiation on the detector;
determining from an analysis of the data a pupil position; and
adjusting a direction of radiation emitted by an illumination source responsive to the determined pupil position in order to substantially center the emitted radiation on the pupil, the illumination source substantially coaxial with the detector and configured to emit a beam of radiation having a diameter less than a pupil diameter.

22. The method recited in claim 21, wherein the illumination source is adapted to emit in the infrared range.

23. The method recited in claim 22, wherein the illumination source is adapted to emit below 1.5 μm.

24. The method recited in claim 21, wherein the illumination source is selected from a group consisting of a monochromatic laser, a light-emitting diode, and superluminescent light-emitting diode, and a resonant-cavity light-emitting diode.

25. The method recited in claim 21, further comprising the step of positioning a beamsplitter to reflect radiation from the illumination source onto the eye and to pass the reflected radiation to the detector, for permitting a substantially coincident path of the emitted radiation and the reflected radiation.

26. The method recited in claim 25, wherein the illumination source is polarized, and wherein the beamsplitter comprises a polarizing beamsplitter.

27. The method recited in claim 21, wherein the illumination source is unpolarized, and further comprising the step of masking specular reflection from the eye from reaching the detector.

28. The method recited in claim 21, wherein the detector comprises a non-imaging detector.

29. The method recited in claim 28, wherein the detector comprises a quadrant detector divided into quarters and having a plurality of concentric, substantially toroidal zones subdivided into quarter-sectors by the quarter divisions.

30. The method recited in claim 29, wherein the determining step comprises, for each quarter, determining an outermost quarter-sector containing reflected radiation and analyzing the data in the outermost quarter-sector only.

31. The method recited in claim 21, wherein the illumination source is unpolarized, and the detector comprises an imaging detector positioned at a focal plane of the illumination source, the generated data comprise pixel data, and the determining step comprises determining from the pixel data the pupil position.

32. The method recited in claim 21, further comprising the step of maintaining an image of the pupil at the detector at a substantially constant size.
